(19) United States
(12) Patent Application Publication
(10) Pub. No.: US 2008/0236596 A1
(43) Pub. Date: Oct. 2, 2008

(54) CUFF SHIELD FOR A PRESSURE CUFF

(75) Inventors: Irvin T. Pierskalla, Prior Lake, MN (US); Kent R. Winger, Prior Lake, MN (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: OPTICAL SENSORS INCORPORATED, Eden Prairie, MN (US)

(21) Appl. No.: 11/695,309

(22) Filed: Apr. 2, 2007

(51) Int. Cl. A61B 5/02; A61F 5/37

(52) U.S. Cl. 128/846; 600/500

(57) ABSTRACT

A cuff shield for a pressure cuff comprises a liquid impervious layer and an absorbent layer. The liquid impervious layer includes an outer surface and an inner surface. The outer surface of the liquid impervious layer has an adhesive portion for removably securing the cuff shield to the pressure cuff. The absorbent layer is secured to the liquid impervious layer and includes an outer surface, an inner surface, and a release strip on the outer surface of the absorbent layer. The release strip on the absorbent layer is configured to serve as a protective cover for an adhesive portion on a second cuff shield. This design of the release strip enables delivering the cuff shields on a roll

### BACKGROUND OF THE INVENTION

The present invention pertains to an infection control device for a pressure cuff. In particular, the present invention pertains to a cuff shield that attaches to an inner surface of a pressure cuff to reduce the spread of biological contaminants between patients.

Toxic shock syndrome, flesh eating bacteria and the bird flu have all created an acute awareness, if not outright panic, regarding the world of infectious diseases. Whether or not this panic is warranted, prevention and treatment of infections and infectious diseases are major concerns for the healthcare industry.

Nosocomial infections, infections that originate in hospitals or health care settings, have been an issue ever since Ignaz Semmelweis discovered in the 1840's that the lack of hand washing lead to a high mortality rate in postpartum women. Since then there has been significant progress, especially in the last 30 years, in the prevention of nosocomial infections. In 1976 the Joint Commission on Accreditation of Healthcare Organizations (JCAHO) published standards for infection control and in 1985 the Center for Disease Control and Prevention (CDC) reported that hospitals with infection control programs had reduced nosocomial infections by one third. Since then, however, the nosocomial infection rate has remained fairly stable at 5 to 6 hospital acquired infections for every 100 hospitalizations as estimated by the CDC's National Nosocomial Infection Surveillance system (NNIS). It is estimated that nosocomial infections cost approximately $4.5 billion and contribute to 88,000 deaths per year, or 1 death every 6 minutes.

Recent research has found that potentially harmful bacteria can be cultured from many surfaces in hospital environments, from computer keyboards to neckties. Medical devices, however, including pressure cuffs, are of particular concern because they regularly come into contact with patients, often without cleaning or some sort of infection control procedure when used from patient to patient. In addition, the health of patients in a hospital or clinic setting is typically already compromised, thereby placing them at further risk for nosocomial infections.

Based on the foregoing, an infection control means should be an integral part of pressure cuffs used in skin perfusion pressure systems. Just as a surgeon would not perform surgery without sterile gloves, healthcare providers should not use a pressure cuff when determining skin perfusion pressure without first using some sort of infection control device. In an effort to lower the nosocomial infection rate to the lowest possible level, infection control precautions are essential.

The prior art includes infection control means attempting to address the problem of transmitting nosocomial infections via pressure cuffs. In particular, single use pressure cuff liners were developed that are temporarily attached to the inner surface of the cuff before being placed on a measurement site of a patient. Also, more complex cuff protectors are found in the prior art. Even with the awareness of the need for a protective measure, the use of these liners or protectors is not widespread. The two main reasons are cost and cumbersome application. Many cuff protectors cover the entirety of the cuff, require a large amount of material, and generally include a fastening means to replace that found on the cuff itself.

Conventional cuff liners are not user friendly and result in waste requiring disposal prior to application of the cuff shield to the cuff. In particular, the adhesive surface on the cuff shield includes a “peel-off” protective strip that covers the adhesive layer until the physician is ready to apply the cuff shield to the pressure cuff. When the physician is ready to take a skin perfusion pressure reading, the cuff shield must be grasped with one hand and the protective strip removed with the other hand, thus resulting in wasted time and, moreover, the protective strip then requires disposal. Other methods may require folding, multiple components, and/or insertion of the cuff into the protector. Accordingly, there exists a need for an infection control device that can be obtained and used quickly and easily, with minimal waste, while providing complete protection of the entire (patient) contact surface of the pressure cuff.

### BRIEF SUMMARY OF THE INVENTION

The present invention solves the foregoing problems by providing a cuff shield for a pressure cuff comprising a liquid impervious layer and an absorbent layer. The liquid impervious layer includes an outer surface and an inner surface. The outer surface of the liquid impervious layer has an adhesive portion for removably securing the cuff shield to the pressure cuff. The absorbent layer is secured to the liquid impervious layer and includes an outer surface, an inner surface, and a release strip on the outer surface of the absorbent layer. The release strip on the absorbent layer is configured to serve as a protective cover for an adhesive portion on a second cuff shield.

An alternate embodiment of the invention is comprised of a liquid impervious layer and an absorbent having openings formed therethrough. The absorbent layer has a release strip on an outer surface. The release strip includes an adhesive portion on an inner surface that is exposed through the openings in the absorbent and impervious layers for removably securing the cuff shield to the pressure cuff. An outer surface of the release strip is configured to serve as a protective cover for the exposed adhesive portion on a second cuff shield.

The present invention also provides a method for applying the cuff shield to the pressure cuff. The method generally includes: providing a dispenser roll having a plurality of cuff shields wound around a core; removing one of the cuff shields from the dispenser roll; aligning the removed cuff shield with an inner surface of the pressure cuff; and attaching the adhesive portion of the liquid impervious layer of the removed cuff shield to the inner surface of the pressure cuff (such that is does not interfere with the cuff fastening means).

### BRIEF DESCRIPTION OF THE DRAWINGS

FIG. 1 is a schematic representation of a perfusion pressure monitor in use with a patient.

FIG. 2 is an exploded perspective view of a cuff shield according to the present invention.

FIGS. 3A and 3B illustrate the cuff shield removably attached to a pressure cuff.

FIG. 4 illustrates an absorbent layer of the cuff shield according to the present invention.

FIG. 5 is a perspective view of a first side of an alternative embodiment of a cuff shield according to the present invention.

FIG. 6 is a perspective view of a second side of the cuff shield embodiment illustrated in FIG. 5.

FIG. 7 is a perspective view of a cuff shield dispenser roll according to the present invention.

FIG. 8 is a diagram illustrating an alternative cuff shield dispensing means according to the present invention.

### DETAILED DESCRIPTION OF THE INVENTION

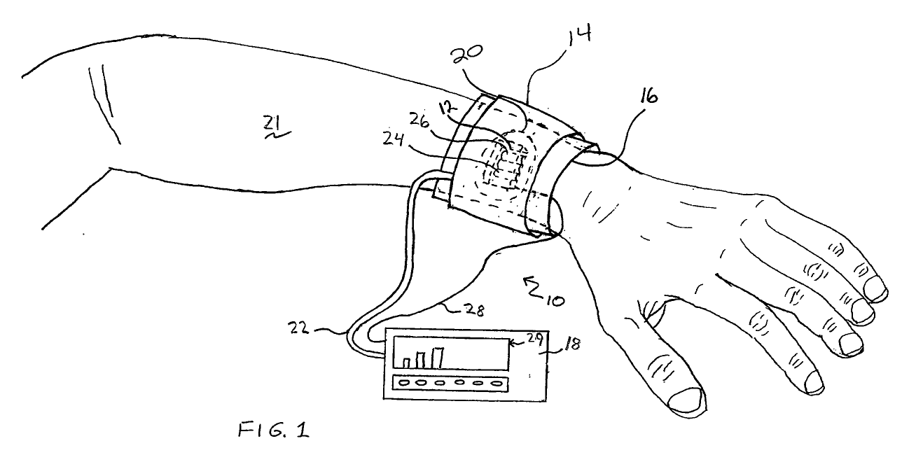

Referring to FIG. 1, a schematic diagram depicting a representative, but not limiting, perfusion pressure monitoring system 10 is illustrated. The skin perfusion pressure monitoring system 10 broadly includes optical probe or sensor 12, pressure cuff 14, cuff shield 16, and skin perfusion pressure instrument 18 with display monitor 20. Optical sensor 12 is positioned underneath pressure cuff 14 proximate the skin of the patient's limb 21. In order to maintain optical sensor 12 at a desired position on the patient's limb, sensor 12 may include an adhesive-type backing to enable temporary attachment of the sensor to the skin.

Cuff shield 16 is disposed between the skin of the patient's limb 21 and an interior surface of pressure cuff 14. In particular, and as will be discussed in more detail to follow, cuff shield 16 is designed to be temporarily attached to pressure cuff 14 in order to avoid direct contact between the patient's skin and pressure cuff 14. As a result, the risk of transmitting various infections between patients is minimized.

Cuff shield 16 of the present invention may be particularly useful, for example, in conjunction with a pressure system such as that described in U.S. patent application Ser. No. 11/468,203, filed Aug. 29, 2006, the entirety of which is hereby incorporated by reference. However, it will be obvious to one skilled in the art that cuff shield 16 may be used with numerous other types of pressure systems and pressure cuffs.

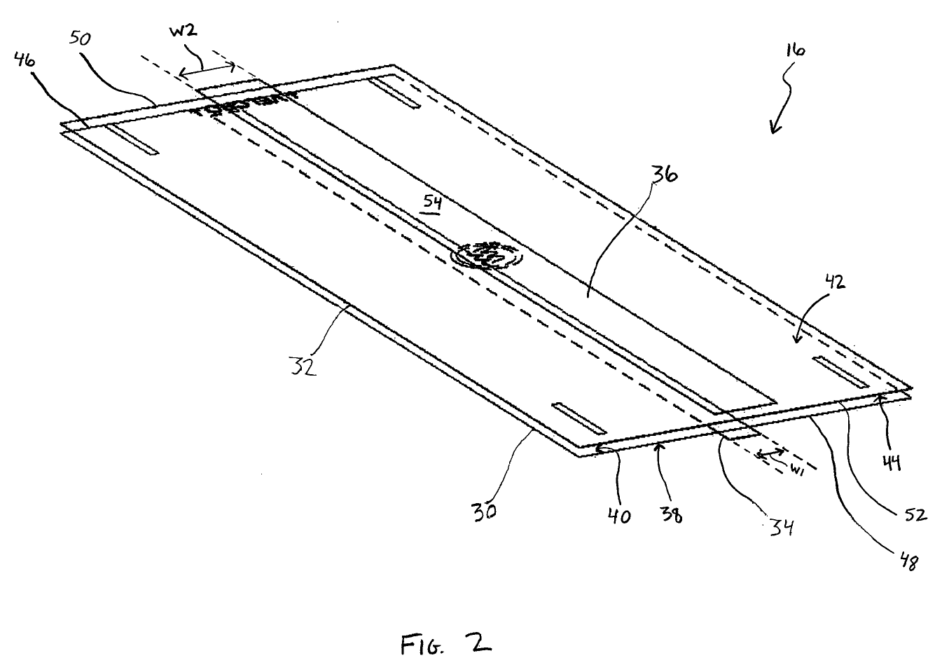

Now that a general overview of one embodiment of a system that takes a measurement using a cuff has been provided, the focus of the discussion will now shift to the inventive design of cuff shield 16. In particular, FIG. 2 is an exploded perspective view of cuff shield 16 according to the present invention, which includes first layer 30, second layer 32, adhesive portion 34, and release strip 36. First layer 30 of cuff shield 16 includes outer surface 38 and inner surface 40. Similarly, second layer 32 of cuff shield 16 includes outer surface 42 and inner surface 44. Although illustrated in an exploded state, cuff shield 16 is manufactured such that inner surface 40 of first layer 30 is bonded to inner surface 44 of second layer 32 to form a single cuff shield with multiple layers.

First layer 30 is preferably formed from a liquid impervious material that prevents fluids from penetrating through the layer. The liquid impervious material may be, for example, a polymer film, although numerous other materials that form a barrier to liquids are also contemplated. Second layer 32 is preferably formed from an absorbent material capable of absorbing and/or wicking away moisture from the patient's skin. The absorbent material may be, for example, a non-woven web, although numerous other materials that have absorbent and/or wicking properties are also contemplated. In addition, second layer 32 may include a plurality of silver fibers disposed within the absorbent material for use as an anti-microbial agent.

Cuff shield 16 is designed to act as a barrier such that moisture on the patient's skin may be absorbed by second layer 32 without penetrating through first layer 30. This type of barrier is effective to minimize the possibility that harmful biological contaminants and bacteria on a patient's body may be transferred to an inner surface of pressure cuff 14, and subsequently, to the body of another patient.

As shown in FIG. 2, adhesive portion 34 has a generally rectangular shape with a width W1. However, adhesive portion 34 may take on numerous other shapes and sizes as would be appreciated by one skilled in the art. In addition, multiple adhesive portions may be used instead of a single adhesive portion 34. Adhesive portion 34 is securable to outer surface 38 of first layer 30, and may be sized so as to extend substantially between first end 46 and second end 48 of first layer 30. In one embodiment, adhesive portion is a pressure sensitive type adhesive that is applied to first layer 30 during the manufacturing process. As will be discussed in further detail to follow, adhesive portion 34 is designed to allow temporary attachment of cuff shield 16 to pressure cuff 14. Therefore, adhesive portion 34 preferably includes adhesive properties that provide a sufficient amount of strength to prevent any substantial movement of cuff shield 16 after attachment to pressure cuff 14, but that also allow for easy removal of cuff shield 16 from pressure cuff 14 after use as will be appreciated by one skilled in the art.

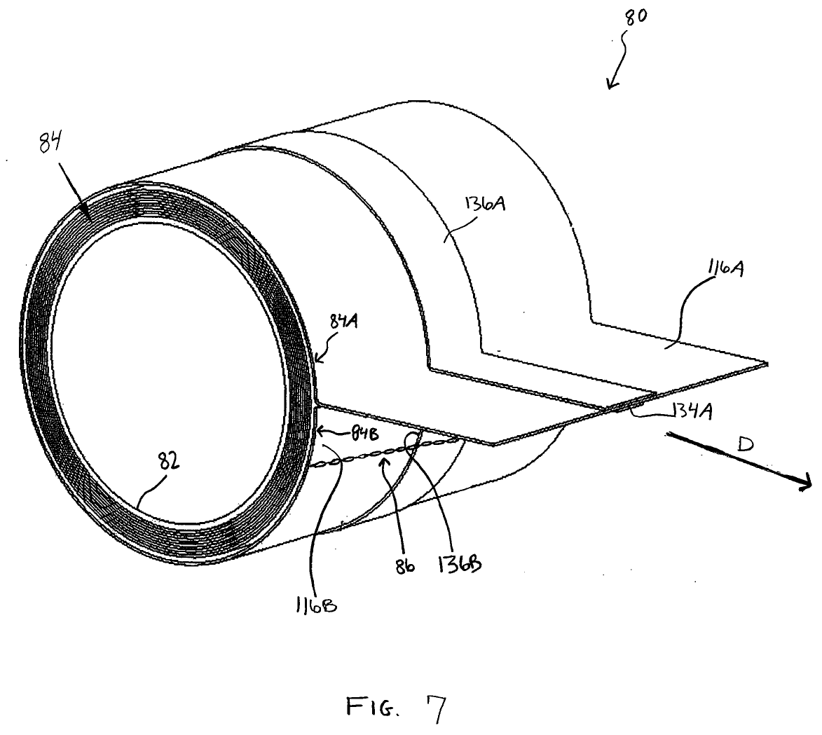

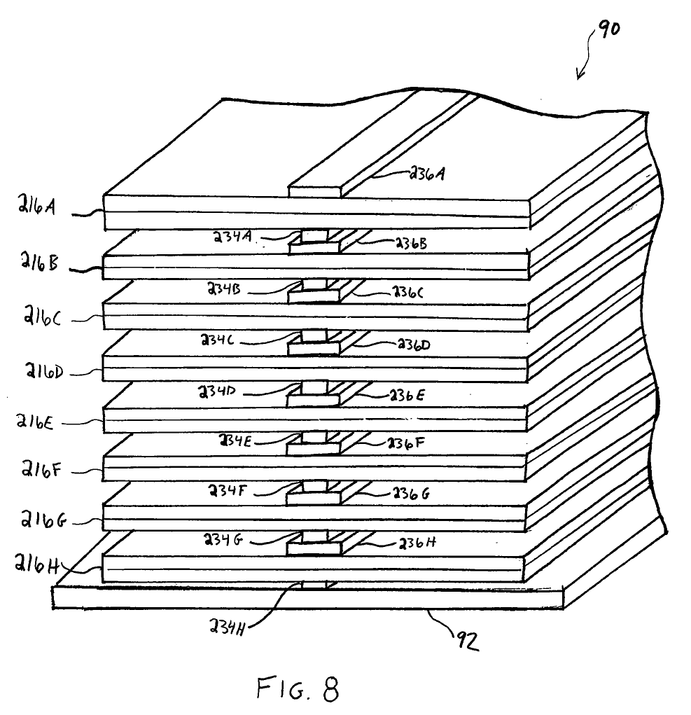

As shown in FIG. 2, release strip 36 also has a generally rectangular shape with a width W2. Release strip 36 may take on numerous other shapes and sizes as would be appreciated by one skilled in the art, but is preferably sized slightly larger and shaped similar to that of adhesive portion 34. Release strip 36 is attachable to outer surface 42 of second layer 32, and may be sized so as to extend substantially between first end 50 and second end 52 of second layer 32. Release strip 36 generally includes a non-adherent surface 54 which is designed to serve as a protective layer for a second cuff shield adjacent cuff shield 16 on a dispenser roll or other dispensing means as will be discussed in reference to FIGS. 7 and 8.

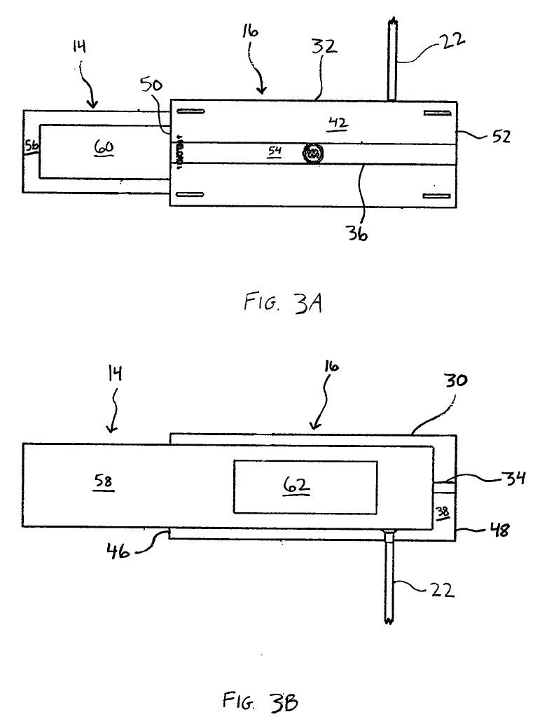

FIGS. 3A and 3B illustrate cuff shield 16 attached to pressure cuff 14. In particular, FIG. 3A is a view of an inner surface of pressure cuff 14 (i.e., the surface that is oriented toward the patient's skin when applying the pressure cuff), while FIG. 3B is a view of an outer surface of pressure cuff 14. As illustrated in FIGS. 3A and 3B, pressure cuff 14 further includes inner surface 56, outer surface 58, first fastening means 60, and corresponding second fastening means 62. First and second fastening means 60 and 62 preferably form a hook-and-loop type fastener, such as the type commonly known as Velcro. However, other suitable fastening means are contemplated that allow the physician to secure pressure cuff 14 once the cuff is wrapped around the patient's limb.

Prior to applying pressure cuff 14 to the patient's limb, cuff shield 16 is first removed from a cuff shield dispenser as will be described in more detail in subsequent paragraphs. In order to temporarily secure the removable cuff shield 16 to pressure cuff 14, outer surface 38 of first layer 30 is first positioned at a desired cuff location with respect to inner surface 56 of pressure cuff 14. Then, adhesive portion 34 on outer surface 38 of first layer 30 is pressed against inner surface 56 of pressure cuff 14 to temporarily adhere cuff shield 16 to pressure cuff 14. Next, the physician wraps pressure cuff 14 around the patient's limb and couples first fastening means 60 to second fastening means 62 as illustrated in FIG. 1. After an SPP measurement is taken as discussed above, cuff shield 16 may be easily removed from pressure cuff 14 and disposed of in accordance with any applicable waste disposal procedures.

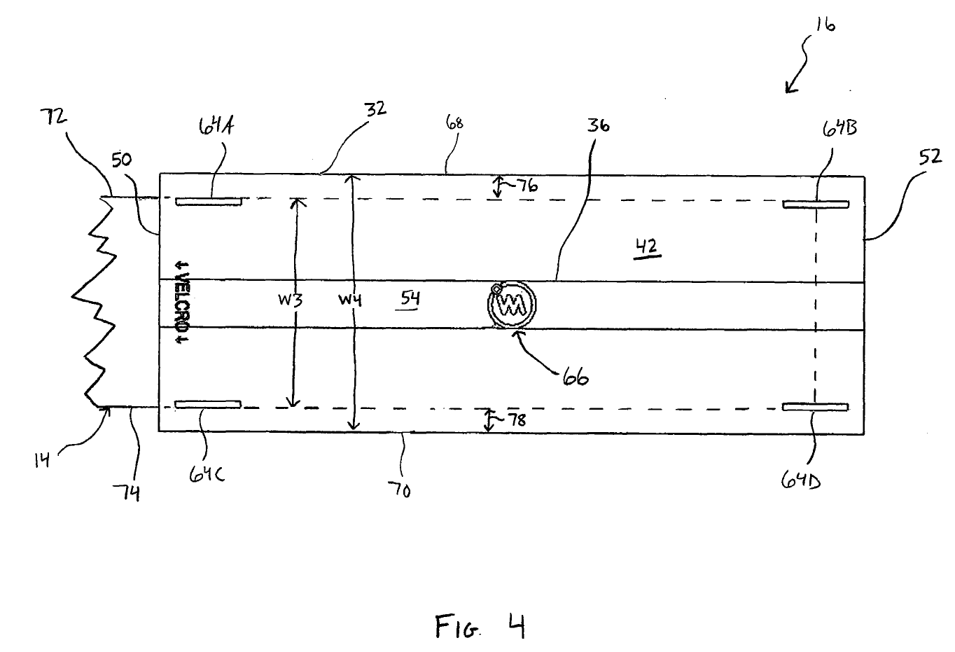

FIG. 4 is a view of cuff shield 16 attached to pressure cuff 14 illustrating a plurality of position markers on second layer 32 including edge markers 64 and sensor marker 66. In particular, cuff shield 16 includes four edge markers 64A-64D located near each of the four corners of second layer 32. Edge markers 64A-64D are designed to assist the physician in properly aligning cuff shield 16 at a desired location with respect to pressure cuff 14 prior to adhering adhesive portion 34 of first layer 30 to inner surface 56 of the pressure cuff. In particular, edge markers 64A-64D ensure that when cuff shield 16 is properly positioned at the desired location with respect to pressure cuff 14, there is no direct contact between inner surface 56 of pressure cuff 14 and the patient's skin when the pressure cuff is secured to the patient's limb. In addition, edge markers 64A-64D may assist in properly placing sensor marker 66 at a position corresponding with the center of the bladder of pressure cuff 14. As shown in FIG. 4, pressure cuff 14 has a width W3 and cuff shield 16 has a larger width W4 such that first and second outer edges 68 and 70 of cuff shield 16 extend past first and second outer edges 72 and 74 of pressure cuff 14 to create first and second overhang regions 76 and 78.

A first set of edge markers 64A and 64B are configured to align with first outer edge 72 of pressure cuff 14, while a second set of edge markers 64C and 64D are configured to align with second outer edge 74 of the pressure cuff. Although one skilled in the art will appreciate that it may be possible to align cuff shield 16 with both first and second outer edges 72 and 74 of pressure cuff 14 with only one set of edge markers, having both first and second sets of edge markers may make the task of aligning cuff shield 16 both easier and faster for the physician. However, it should be understood that embodiments of cuff shield 16 that include only one set of edge markers are contemplated and within the intended scope of the present invention.

One skilled in the art will also appreciate that although the foregoing discussion focused on “sets” of edge markers, a single edge marker may be used to properly position and align cuff shield 16 with an outer edge of pressure cuff 14. For example, a single edge marker 64A may be sufficient to align cuff shield 16 with first outer edge 72 of pressure cuff 14. However, having multiple edge markers near opposing corners of first outer edge 68 of cuff shield 16 (i.e., edge markers 64A and 64B) helps the physician to ensure that first outer edge 68 of cuff shield 16 is substantially parallel with first outer edge 72 of pressure cuff 14 across the entire length of the cuff shield. As a result, the possibility that the physician will apply cuff shield 16 to pressure cuff 14 in a “crooked” fashion (i.e., wherein first outer edge 68 of cuff shield 16 is not substantially parallel with first outer edge 72 of pressure cuff 14) is greatly reduced.

Therefore, while it is desirable to have an edge marker near each corner of second layer 32 for many reasons, including those stated above, such a configuration is not necessary to achieve proper placement of cuff shield 16 at the desired location with respect to pressure cuff 14. Thus, embodiments of cuff shield 16 that include a larger or smaller number of edge markers, as well as edge markers placed at various other locations on second layer 32 of the cuff shield, are also contemplated.

Edge markers 64A-64D are shown in FIG. 4 as having a generally rectangular configuration, although numerous other configurations are also contemplated. However, configurations that include a linear side (such as a rectangle) may be preferred due to their ease of alignment with outer edges 72 and 74 of pressure cuff 14.

As shown in FIG. 4, sensor marker 66 is preferably placed at a generally central location on second layer 32 of cuff shield 16 (i.e., a location that is generally equidistant between first end 50 and second end 52 in the direction of the length and between first outer edge 68 and second outer edge 70 in the direction of the width). Sensor marker 66 is preferably sized and shaped similar to sensor 12 of FIG. 1. However, various other sizes, shapes, and locations of sensor marker 66 are also contemplated.

Sensor marker 66 is designed to align with sensor 12 after sensor 12 is positioned on the patient's limb as discussed above in reference to FIG. 1. In particular, sensor marker 66 provides a means for centering sensor 12 underneath the bladder portion of pressure cuff 14. Although sensor marker 66 is not a necessary component of the present invention, the sensor marker is desirable because it allows for improved SPP measurements by providing a repeatable method for accurate sensor placement underneath pressure cuff 14.

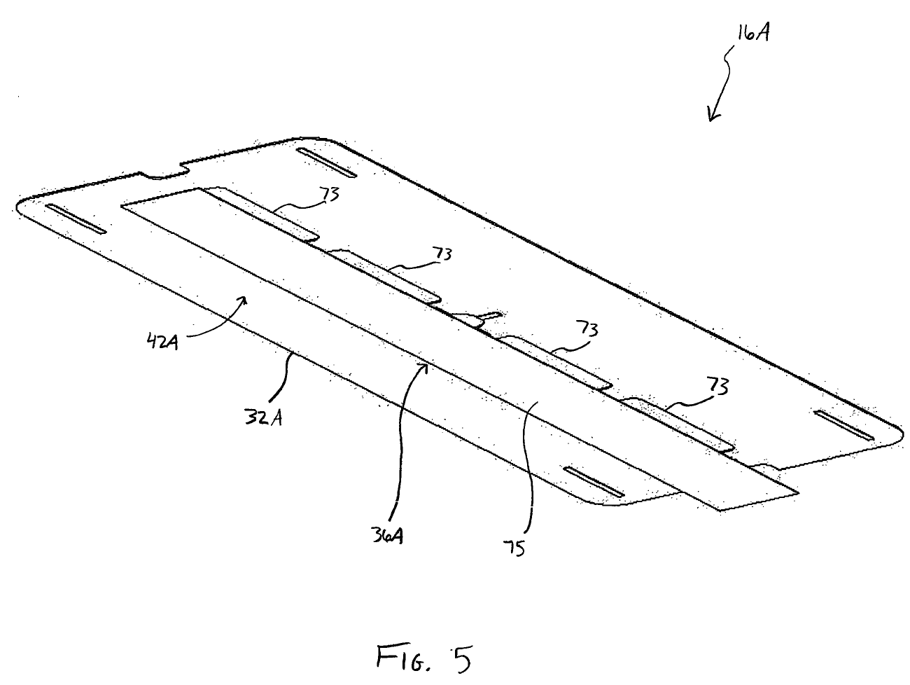

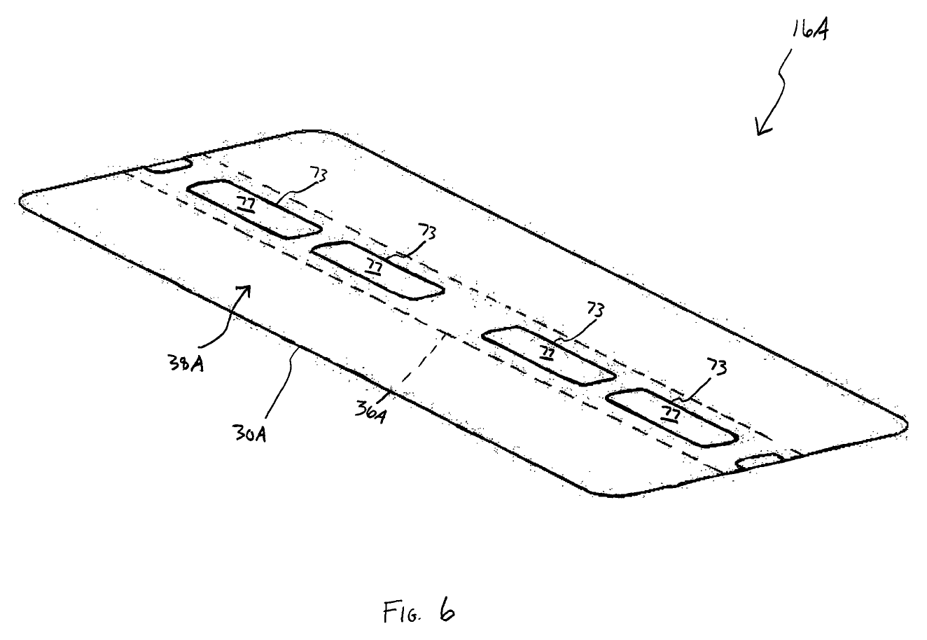

FIGS. 5 and 6 illustrate an alternative embodiment of a cuff shield according to the present invention. In particular, FIG. 5 is a perspective view of a first side of cuff shield 16A, while FIG. 6 is a perspective view of a second side of the cuff shield. As shown in FIGS. 5 and 6, cuff shield 16A is similar to cuff shield 16 described in reference to the above figures, but further includes a plurality of openings 73 extending through both first layer 30A and second layer 32A. Release strip 36A includes outer surface 75 and inner surface 77. An adhesive is applied to inner surface 77, which functions to couple inner surface 77 of release strip 36A to outer surface 42A of second layer 32A. In addition, the adhesive on inner surface 77 is exposed through openings 73 in first and second layers 30A and 32A, allowing the physician to removably secure cuff shield 16 to pressure cuff 14 as previously described. Thus, one skilled in the art will appreciate that the need for a separate adhesive portion, such as adhesive portion 34 shown in FIG. 2, is eliminated.

FIG. 7 is a perspective view of cuff shield dispenser roll 80 according to the present invention. Dispenser roll 80 includes core 82 and a plurality of successively stacked layers 84 wound around core 82, the stacked layers 84 being formed from cuff shields 116. As shown in FIG. 7, first layer 84A includes cuff shield 116A, and second layer 84B includes cuff shield 116B. Cuff shield 116A is attached to cuff shield 116B at perforated connection 86. One skilled in the art will appreciate that although cuff shields 116A and 116B have a design similar to that of cuff shield 16, dispenser roll 80 may be formed using other cuff shield designs, such as cuff shield 16A shown and described in reference to FIGS. 5 and 6, without departing from the intended scope of the present invention.

Dispenser roll 80 is designed such that the adhesive portion of a cuff shield on a first layer is protected by the release strip on a cuff shield in a second layer adjacent to the first layer. As shown in FIG. 7, adhesive portion 134A on first cuff shield 116A in first layer 84A is protected by release strip 136B on second cuff shield 116B in second layer 84B prior to removing first cuff shield 116A from dispenser roll 80. Thus, when first cuff shield 116A is removed from dispenser roll 80, the physician may apply it directly to a pressure cuff without having to first remove any other protective layer to expose adhesive portion 134A.

As shown in FIG. 7, consecutive cuff shields 116 are attached to one another by a perforated connection 86. Thus, for example, when the physician removes cuff shield 116A from dispenser roll 80, the physician pulls the cuff shield in direction D until it is “peeled” from the roll. Then, the physician separates cuff shield 116A from cuff shield 116B at perforated connection 86 by a tearing or similar motion. Cuff shield 116A may then be applied to a pressure cuff as discussed above in reference to FIGS. 1-4. Therefore, one skilled in the art will appreciate that dispenser roll 80 provides cuff shields which may be removed and applied to a pressure cuff quickly and easily, with no additional waste that requires disposal.

The cuff shields forming the successively stacked layers 84 of dispenser roll 80 are shown without edge markers 64 or sensor marker 66 for purposes of example and not for limitation. Therefore, it should be understood that dispenser roll 80 may alternatively include cuff shields having either edge markers 64, sensor marker 66, or both. Furthermore, although not depicted in FIG. 7, dispenser roll 80 may be designed to fit onto a dispenser roll holder. Such a holder may be similar to, for example, a paper towel holder or a holder for disposable hand towels commonly found in public restrooms. However, those skilled in the art will appreciate that dispenser roll 80 may be used in numerous other types of dispenser roll holders without departing from the intended scope of the present invention.

FIG. 8 is a diagram illustrating an alternative cuff shield dispensing means according to the present invention. In particular, cuff shield dispensing means 90 includes a plurality of cuff shields 216A-216H stacked vertically in layers on top of base 92. Cuff shield dispensing means 90 is designed such that the adhesive portion of a cuff shield on a first layer is protected by the release strip on a cuff shield in a second layer adjacent to the first layer. Thus, as shown in FIG. 8, adhesive portion 234A on first cuff shield 216A is protected by release strip 236B on second cuff shield 216B prior to removing first cuff shield 216A from the stack of cuff shields. Thus, when first cuff shield 216A is removed from dispensing means 90, the physician may apply it directly to a pressure cuff without having to first remove any other protective layer to expose adhesive portion 234A.

Once again, cuff shields 216A-216H are shown without edge markers 64 or sensor marker 66 for purposes of example and not for limitation. Therefore, it should be understood that dispensing means 90 may alternatively include cuff shields having either edge markers 64, sensor marker 66, or both. In addition, although dispensing means 90 is depicted as including eight cuff shields stacked on top of base 92, dispensing means 90 may include any number of cuff shields without departing from the intended scope of the present invention. One skilled in the art will also appreciate that although cuff shields 216A-216H have a design similar to that of cuff shield 16, dispenser means 90 may alternatively be formed using other cuff shield designs, such as cuff shield 16A shown and described in reference to FIGS. 5 and 6.The cuff shield embodiments in the foregoing figures were shown and described with reference to a pressure cuff for a perfusion pressure monitoring system for purposes of example only. One skilled in the art will appreciate that cuff shields in accordance with the present invention may be used as protective barriers with other types of pressure cuffs such as, for example, sphygmomanometer cuffs.

Although the present invention has been described with reference to preferred embodiments, workers skilled in the art will recognize that changes may be made in form and detail without departing from the spirit and scope of the invention.

I claim:

1. A cuff shield for a pressure cuff comprising:
a liquid impervious layer having an outer surface and an inner surface, the outer surface of the liquid impervious layer having an adhesive portion for removably securing the cuff shield to the pressure cuff; and
an absorbent layer coupled to the liquid impervious layer and having an outer surface, an inner surface, and a release strip on the outer surface of the absorbent layer, wherein the release strip is configured to serve as a protective cover for an adhesive portion on a second cuff shield.

2. The cuff shield of claim 1, wherein the liquid impervious layer is formed from a polymer film.

3. The cuff shield of claim 1, wherein the absorbent layer is formed from a non-woven web.

4. The cuff shield of claim 1, further comprising edge markers on the outer surface of the absorbent layer for indicating proper alignment of the cuff shield with the pressure cuff.

5. The cuff shield of claim 1, further comprising a sensor marker on the outer surface of the absorbent layer for indicating the desired placement of a sensor with respect to the pressure cuff.

6. The cuff shield of claim 1, wherein the adhesive portion extends substantially between a first end and a second end of the liquid impervious layer.

7. The cuff shield of claim 6, wherein the release strip extends substantially between a first end and a second end of the absorbent layer.

8. The cuff shield of claim 1, wherein the release strip includes an outer surface and an inner surface, the inner surface of the release strip having an adhesive material for bonding the inner surface of the release strip to the absorbent layer.

9. The cuff shield of claim 8, further comprising one or more openings extending through the absorbent layer and the liquid impervious layer, wherein the adhesive portion is formed from the adhesive material exposed through the one or more openings in the absorbent and liquid impervious layers.

10. A cuff shield for a pressure cuff comprising:
a first layer having an outer surface and an inner surface, the outer surface of the first layer having an adhesive portion for removably securing the cuff shield to the pressure cuff at a desired cuff location; and
a second layer secured to the first layer and having an inner surface, an outer surface, and a release strip on the outer surface of the second layer configured to serve as a protective cover for an adhesive portion on a second cuff shield, the outer surface of the second layer comprising at least one position marker for properly positioning the cuff shield at the desired cuff location.

11. The cuff shield of claim 10, wherein the first layer is a liquid impervious layer.

12. The cuff shield of claim 11, wherein the first layer is formed from a polymer film.

13. The cuff shield of claim 10, wherein the second layer is an absorbent layer.

14. The cuff shield of claim 13, wherein the second layer is formed from a non-woven web.

15. The cuff shield of claim 10, wherein the at least one position marker on the outer surface of the second layer comprises at least one edge marker for aligning the cuff shield with one or more outer edges of the pressure cuff.

16. The cuff shield of claim 10, wherein the at least one position marker on the outer surface of the second layer comprises a sensor marker for indicating the desired placement of a sensor with respect to the pressure cuff.

17. A method for applying disposable cuff shields to a pressure cuff, the method comprising:
providing a dispenser roll having a plurality of cuff shields, each cuff shield comprising:
a liquid impervious layer having an outer surface and an inner surface, the outer surface of the liquid impervious layer having an adhesive portion for removably securing the cuff shield to the pressure cuff; and
an absorbent layer coupled to the liquid impervious layer and having an outer surface, an inner surface, and a release strip on the outer surface of the absorbent layer, wherein the release strip is configured to serve as a protective cover for an adhesive portion on a second cuff shield;
removing one of the cuff shields from the dispenser roll;
aligning the removed cuff shield with an inner surface of the pressure cuff; and
attaching the adhesive portion of the liquid impervious layer of the removed cuff shield to the inner surface of the pressure cuff.

18. The method of claim 17, wherein the outer surface of the absorbent layer further comprises one or more edge markers for aligning the cuff shield at a desired location on the inner surface of the pressure cuff.

19. The method of claim 18, wherein the step of aligning the removed cuff shield with the inner surface of the pressure cuff further comprises aligning the edge markers with one or more outer edges of the pressure cuff.

20. The method of claim 17, wherein the removed cuff shield is aligned such that it does not interfere with a cuff fastening means on the pressure cuff.

21. A cuff shield dispenser roll comprising a plurality of successively stacked layers wound around a core, the successively stacked layers formed from cuff shields for a pressure cuff, each cuff shield comprising:
a liquid impervious member having an outer surface and an inner surface, the outer surface of the liquid impervious member having an adhesive portion for removably securing the cuff shield to the pressure cuff; and
an absorbent member secured to the liquid impervious member and having an inner surface, an outer surface, and a release strip bonded to the outer surface of the absorbent member, wherein the release strip is configured to serve as a protective cover for an adhesive portion on another cuff shield positioned in an adjacent one of the successively stacked layers.

22. The cuff shield dispenser roll of claim 21, wherein each cuff shield includes at least one end adjoined to an end of another one of the cuff shields.

23. The cuff shield dispenser roll of claim 22, wherein adjoined ends of consecutive cuff shields have a perforated connection.
